(19) United States
(12) Patent Application Publication
(10) Pub. No.: US 2011/0184258 A1
(43) Pub. Date: Jul. 28, 2011

(54) Balloon Catheter Analyte Measurement Sensors and Methods for Using the Same

(75) Inventor: Gary Ashley Stafford, Hayward, CA (US)

(73) Assignee: Abbott Diabetes Care Inc., Alameda, CA (US)

(21) Appl. No.: 13/011,918

(22) Filed: Jan. 23, 2011

Related U.S. Application Data
(60) Provisional application No. 61/299,340, filed on Jan. 28, 2010.

(51) Int. Cl. A61B 5/145

(52) U.S. Cl. 600/309

(57) ABSTRACT

Balloon catheters, assemblies and systems and methods for using them for the continuous in vivo monitoring of one or more selected analytes or other physiological chemistries within a patient using an implantable analyte sensor associated with a balloon catheter are provided.

### RELATED APPLICATION

The present application claims the benefit of U.S. provisional patent application No. 61/299,340 filed Jan. 28, 2010, entitled “Balloon Catheter Analyte Measurement Sensors and Methods for Using the Same”, the disclosure of which is incorporated herein by reference in its entirety for all purposes.

### BACKGROUND

There are a number of instances when it is desirable or necessary to monitor the concentration of an analyte, such as glucose, lactate, or oxygen, for example, in bodily fluid of a body. For example, it may be desirable to monitor high or low levels of glucose in blood or other bodily fluid that may be detrimental to a human. In a healthy human, the concentration of glucose in the blood is maintained between about 0.8 and about 1.2 mg/mL by a variety of hormones, such as insulin and glucagons, for example. If the blood glucose level is raised above its normal level, hyperglycemia develops and attendant symptoms may result. If the blood glucose concentration falls below its normal level, hypoglycemia develops and attendant symptoms, such as neurological and other symptoms, may result. Both hyperglycemia and hypoglycemia may result in death if untreated. Maintaining blood glucose at an appropriate concentration is thus a desirable or necessary part of treating a person who is physiologically unable to do so unaided, such as a person who is afflicted with diabetes mellitus.

Certain compounds may be administered to increase or decrease the concentration of blood glucose in a body. By way of example, insulin can be administered to a person in a variety of ways, such as through injection, for example, to decrease that person's blood glucose concentration. Further by way of example, glucose may be administered to a person in a variety of ways, such as directly, through injection or administration of an intravenous solution, for example, or indirectly, through ingestion of certain foods or drinks, for example, to increase that person's blood glucose level.

Regardless of the type of adjustment used, it is typically desirable or necessary to determine a person's blood glucose concentration before making an appropriate adjustment. Typically, blood glucose concentration is monitored by a person or sometimes by a physician using an in vitro test that requires a blood sample. The person may obtain the blood sample by withdrawing blood from a blood source in his or her body, such as a vein, using a needle and syringe, for example, or by lancing a portion of his or her skin, using a lancing device, for example, to make blood available external to the skin, to obtain the necessary sample volume for in vitro testing. Typically, blood extraction for in vitro glucose testing is accomplished by way of a “finger stick” since the tissue of the fingertip is highly perfused with blood vessels. There are other commercially available glucose monitoring systems that allow for sample extraction from sites other than the finger, such as from the surface of a palm, a hand, an arm, a thigh, a leg, the torso, or the abdomen. The fresh blood sample, wherever obtained, is then applied to an in vitro testing device, such as an analyte test strip, whereupon suitable detection methods, such as calorimetric, electrochemical, or photometric detection methods, may be used to determine the person's actual blood glucose level. The foregoing procedure provides a blood glucose concentration for a particular or discrete point in time, and thus, must be repeated periodically, in order to monitor blood glucose over a longer period.

While existing analytical techniques and devices for in vitro glucose measurements have a high level of accuracy, there are disadvantages to these existing options. First, sampling even a minimal amount of blood multiple times per day is associated with risks of infection, nerve and tissue damage, and discomfort to the patient. Second, in the case of dynamic changes in glucose concentration, very frequent or even continuous measurements of blood glucose levels are required. Having a real-time provision of a patient's blood glucose level may be particularly necessary in an emergent, urgent or intensive care situation where changes in a patient's blood glucose level may have immediate detrimental effects. This may be particularly so during surgical operations or in post-operative situations. Moreover, continuous monitoring is generally more desirable than discrete or periodic testing in that it may provide a more comprehensive assessment of glucose levels and more useful information, including predictive trend information.

There are available continuous blood glucose monitoring systems which include sensors configured to be at least partially implantable in a patient's body. Examples of such in vivo blood glucose monitoring systems, which are designed to provide continuous or semi-continuous in vivo measurement of an individual's glucose concentration, are disclosed in U.S. Pat. Nos. 6,175,752; 6,284,478; 6,134,461; 6,560,471; 6,746,582; 6,579,690; 6,932,892; and 7,299,082; incorporated by reference herein. These in vivo sensors may be configured to be placed in substantially continuous contact with a blood source, e.g., a blood vessel, or alternatively, in substantially continuous contact with bodily fluid other than blood, such as dermal or subcutaneous fluid.

Other types of in vivo sensors, such as intravascular wires and catheters have also been used for continuous monitoring of biological analytes and various physiological parameters including, but not limited to, localized temperature, blood pressure, blood flow, etc. However, it may be advantageous to configure a similar sensor and system for the continuous monitoring of analyte and other biological chemical levels.

Notwithstanding the efforts in the art, there remains a need for in vivo sensors which provide useful blood glucose or other physio-chemical readings for an extended period of time, particularly in patients in emergent, urgent, surgical and post-operative situations.

### INCORPORATION BY REFERENCE

The following patents, applications and/or publications are incorporated herein by reference for all purposes: U.S. Pat. Nos. 4,545,382; 4,711,245; 5,262,035; 5,262,305; 5,264,104; 5,320,715; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,167,818; 7,299,082; and 7,866,026; U.S. Published Application Nos. 2004/0186365; 2005/0182306; 2006/0025662; 2006/0091006; 2007/0056858; 2007/0068807; 2007/0095661; 2007/0108048; 2007/0199818; 2007/0227911; 2007/0233013; 2008/0066305; 2008/0081977; 2008/0102441; 2008/0148873; 2008/0161666; 2008/0267823; 2009/0054748; 2009/0247857; 2009/0294277; 2010/0081909; 2010/0198034; 2010/0213057; 2010/0230285; 2010/0313105; 2010/0326842; and 2010/0324392; U.S. patent application Ser. Nos. 12/807,278; 12/842,013; and 12/871,901; and U.S. Provisional Application Nos. 61/238,646; 61/246,825; 61/247,516; 61/249,535; 61/317,243; 61/345,562; and 61/361,374.

### SUMMARY

The present disclosure is generally directed to the continuous in vivo monitoring of one or more selected chemical species or agents, including analytes and/or physio-chemical parameters, within a patient using an implantable analyte sensor associated with a balloon catheter. The disclosure provides catheters, catheter assemblies and catheter systems as well as methods for using them.

In one embodiment, subject catheter or catheter assembly comprises an in vivo analyte sensor which is positionable endovascularly or intravascularly or otherwise within the patient's body, e.g., within an organ or other portion of the body containing bodily fluid, to continuously monitor analyte levels. The subject catheter or catheter assembly may include a balloon catheter which comprises an elongated, thin-walled tube with an expandable or inflatable balloon positioned at a distal end or portion of the tube. The sensor portion of the catheter includes one or more sensors positioned for contact with the bodily fluid. In one aspect, the sensors are associated with the balloon as being provided on an exterior surface of the balloon, an interior surface of the balloon, and/or at the distal portion of the catheter tubing adjacent to the balloon.

The subject catheters, assemblies and systems may be used in the context of a therapeutic and/or diagnostic catheter-based procedure in which at least one analyte-sensing catheter is used to continuously monitor a patient's analyte level, either prior to, during and/or subsequent to the underlying procedure. In some embodiments, the analyte-sensing catheter is an ancillary or discrete component of a catheter system which further includes other endovascular/intravascular components, including but not limited to one or more other catheters for performing primary diagnostic and/or therapeutic procedures. With such embodiments, the analyte sensing catheter may be transvascularly positioned at a target site within the patient's vasculature prior to, during and/or after the procedure. In other embodiments, the analyte-sensing functions are integrated into a catheter or catheter assembly configured for performing one or more therapeutic and/or diagnostic procedures, i.e., a multi-functional catheter. In such embodiments, the analyte-sensor catheter may be maintained within the vasculature throughout the procedure. When it is desired to monitor the target analyte(s) subsequent to the procedure, the subject analyte-sensing catheter (whether configured as a single-function or a multi-function catheter) may be left within the patient's vasculature for a discrete amount of time, such as while the patient is in recovery or in intensive care immediately after a surgical procedure to monitor, for example, blood cell counts, toxic blood levels, etc. In some cases, protocol requires that a surgical procedure only be performed when one or more certain target analytes are in predefined or “safe” ranges. As such, the subject analyte-sensing catheter(s) may be intravascularly delivered prior to the intended surgical procedure as a means of assessing when surgery may safely be performed or to monitor poison levels, drug overdose or radiation levels. Depending on the situation, the analyte-sensing catheter may remain within a patient over a time period which may range from minutes, hours, days, weeks, or longer.

The subject catheter assemblies or systems may further comprise a delivery and/or guiding catheter or other guiding components, e.g., a guide wire, and other catheters or instruments deliverable with a catheter for performing a diagnostic and/or therapeutic catheter-based procedure at a target location within the vasculature.

Embodiments of the subject systems may further include analyte monitoring devices which include any one or more of a control unit, transmitter, receiver, transceiver, processor, etc. either coupled directly (e.g., by electrical connections) or remotely (e.g., by wireless connections) to the analyte sensor.

Other embodiments of the present disclosure further provide methods for using the subject catheters, catheter assemblies and catheter systems, as well as methods for continuously monitoring selected analyte levels in a patient. Embodiments of the subject methods further include monitoring one or more target analytes prior to, in conjunction with, or subsequent to a catheter-based or minimally invasive procedure. Such procedures include, but are not limited to, percutaneous transluminal coronary angioplasty (PTCA), percutaneous transluminal embolectomy, directional coronary atherectomy (DCA), cardiac tissue ablation to treat atrial fibrillation, and coronary/vascular stenting.

In one particular application, the present embodiments are directed to continuous monitoring of blood glucose, however, the invention may be applied to any analyte monitoring or measurement application.

These and other features, objects and advantages of the present disclosure will become apparent to those persons skilled in the art upon reading the details of the invention as more fully described below.

### BRIEF DESCRIPTION OF THE DRAWINGS

A detailed description of various aspects, features and embodiments of the present disclosure is provided herein with reference to the accompanying drawings, which are briefly described below. The drawings are illustrative and are not necessarily drawn to scale, with some components and features being exaggerated for clarity. A reference numeral, letter, and/or symbol that is used in one drawing to refer to a particular element or feature maybe used in another drawing to refer to a like element or feature. Included in the drawings are the following:

FIG. 1A is a schematic representation of an analyte monitoring system of the present disclosure, which includes a catheter assembly operatively coupled to a monitoring device;

FIG. 1B is a cross-sectional view taken along line B-B of a distal portion of the catheter assembly of FIG. 1A;

FIG. 2A is a side view of an exemplary embodiment of a balloon portion of a catheter assembly of an analyte monitoring system;

FIG. 2B is a cross-sectional view taken along line B-B of FIG. 2A;

FIG. 2C is an alternative analyte sensor configuration of the balloon portion of FIG. 2A;

FIG. 3A is a side view of another exemplary embodiment of a balloon portion of a catheter assembly of an analyte monitoring system;

FIG. 3B is a cross-sectional view taken along line B-B of FIG. 3A;

FIG. 3C is an alternative analyte sensor configuration of the balloon portion of FIG. 3A; and

FIG. 3D is a further alternative analyte sensor configuration of the balloon portion of FIG. 3A.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. For the purposes of the present application, “downstream” means in the direction of normal blood or body fluid flow through a vessel or organ, e.g., further from the heart in the arterial system, and closer to the heart in the venous system. “Upstream” means in the direction opposite of the downstream direction. References herein to the “proximal” direction, means in the direction toward the end of the device that is closest to and held or manipulated by the physician, while “distal” means in the direction away from the user, and opposite the proximal direction. The terms “chemical species” and “chemical agent” are used interchangeably herein and encompass analytes, physiological chemistries, biological chemistries, etc. that may be contained within any bodily fluid.

Although any methods and materials similar or equivalent to those described herein can also be used in the practice or testing of the present invention, the preferred methods and materials are described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

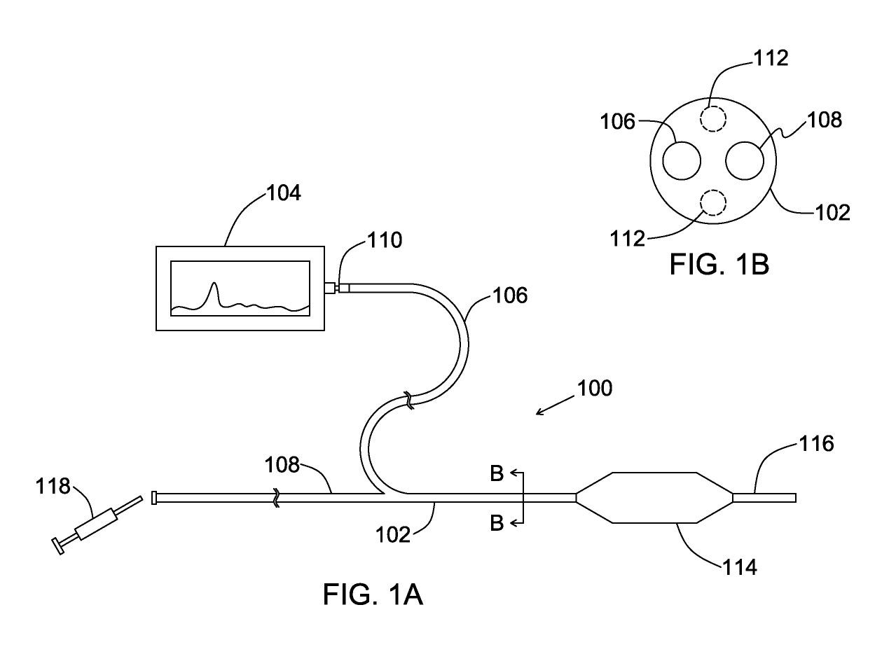

The invention is now described in greater detail with respect to the embodiments of FIGS. 1-3; however, such embodiments are merely exemplary.

FIG. 1A schematically illustrates an chemical species or analyte monitoring system 100 of the present invention, including a balloon catheter assembly operatively coupled to an analyte monitor 104. The catheter assembly includes a multi-lumen distal portion 102 which extends to a distally positioned inflatable or expandable balloon 114. An atraumatic distal tip 116 extends distally from the distal end of balloon 114 which minimizes the chance of tissue damage during delivery of the balloon catheter to a target site within the body of a patient.

As illustrated in the cross-sectional view of FIG. 1B, catheter distal portion 102 includes at least a sensor or monitoring lumen 106 and a balloon inflation/expansion lumen 108. Each of the lumens 106, 108 extend proximally from distal portion 102 into separate proximal tubing portions. Balloon inflation/expansion lumen 108 establishes a fluid communication between the interior of balloon 114 and an inflation/expansion source 118. In one aspect, the inflation/expansion source 118 may be a syringe (as illustrated) filled with a fluid, e.g., saline, or alternatively, a pneumatic supply. Sensor/monitoring lumen 106 extends between a distally positioned analyte sensor (not shown) to a connector 110 which electronically couples the analyte sensor to analyte monitor 104 via wires or the like extending between the sensor and the connector 110.

Analyte or chemical agent/species monitor 104 may include various components for processing and transmitting data regarding the targeted analyte(s) or chemical agent(s)/specie(s), including but not limited to one or more of a display, user interface, control unit, transmitter, receiver, transceiver, processor, etc. With wireless monitor-sensor systems, a transmitter and power supply (e.g., battery) are housed with the sensor device, for example on the balloon portion of the catheter, eliminating the need for a designated sensor monitoring lumen 106. In either embodiment, analyte monitor 104 may be configured to display digital data, e.g., a discrete analyte value, and/or analog data, e.g., a continuous graphical output. Example analyte monitors for use with embodiments of the present disclosure are disclosed in U.S. Pat. Nos. 5,262,035; 5,264,104; 5,262,305; 5,320,715; 5,593,852; 6,175,752; 6,650,471; 6,746,582; and 7,811,231, the disclosures of each of which are incorporated herein by reference for all purposes. In other embodiments, the functions of analyte monitor 104 may be integrated into a monitor configured for measuring and monitoring a patient's vital signs including, but not limited to, blood pressure, pulse, respiration rate, etc. Such an integrated embodiment reduces the number of monitors needed and streamlines space around the patient.

Referring back to FIG. 1B, the multi-lumen portion 102 of the catheter assembly may include one or more lumens 112 configured for performing functions such as perfusion, drainage, venting, etc. and for the delivery of tools and instruments such as a guide wire, probes, etc. in addition to the inflation/expansion lumen 108 and monitoring lumen 106. The lumens 108, 106, 112 may each have their own luminal axis, as illustrated, or two or more of the lumens may be co-axial. The catheter multi-lumen portion 102 may be provided with transverse holes (not shown) within its walls which are in fluid communication with the axial lumens 112 to allow fluid passage from within the respective one or more of its lumens to within the body, or vise-versa. For example, a lumen 112 may be utilized to perfuse contrast medium to within a vessel or organ to enhance visualization thereof by fluoroscopy. Alternatively, one or more of the lumens 112 may have a distal portion which is directed outward through the catheter wall, e.g., curves laterally outward, to allow passage of a tool or instrument to a location within the body for performing a procedure or delivering an agent or the like.

The balloon 114 (FIG. 1A) material may include elastomeric and/or inelastic material, depending on the application and desired function. Elastomeric balloon materials are referred to as compliant. While elastomeric materials are generally soft and conformable, they lack strength and exhibit continuous diameter growth with the application of increasing inflation pressure until rupture occurs. Inelastic balloon materials are referred to as non-compliant or semi-compliant depending on their stiffness. Due to their stiffness, inelastic balloon materials are not soft and conformable. They tend to have very predictable diameter growth characteristics, and distend very little beyond their intended diameter with the application of increasing inflation pressure. Balloons made of these materials, such as angioplasty balloons, are carefully wrapped into a small cross-sectional configuration prior to introduction into the patient. During inflation, the balloons unwrap and assume their intended diameters. During subsequent deflation, however, the balloons do not return to their initial small cross-sectional state.

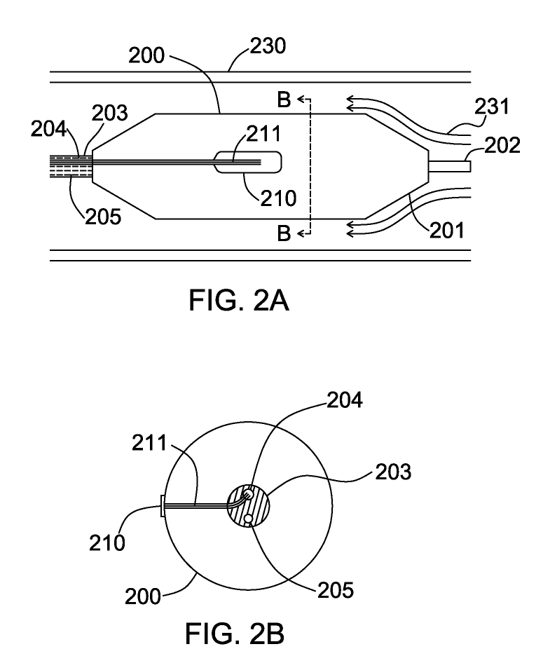

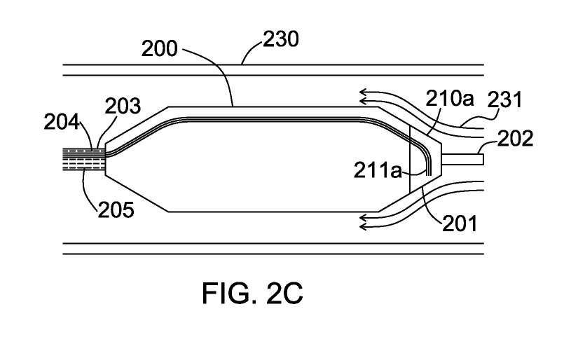

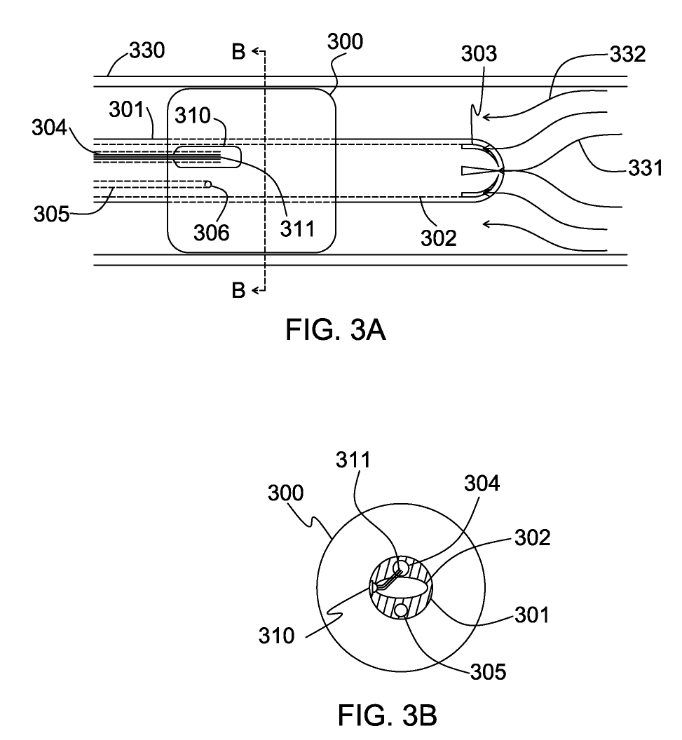

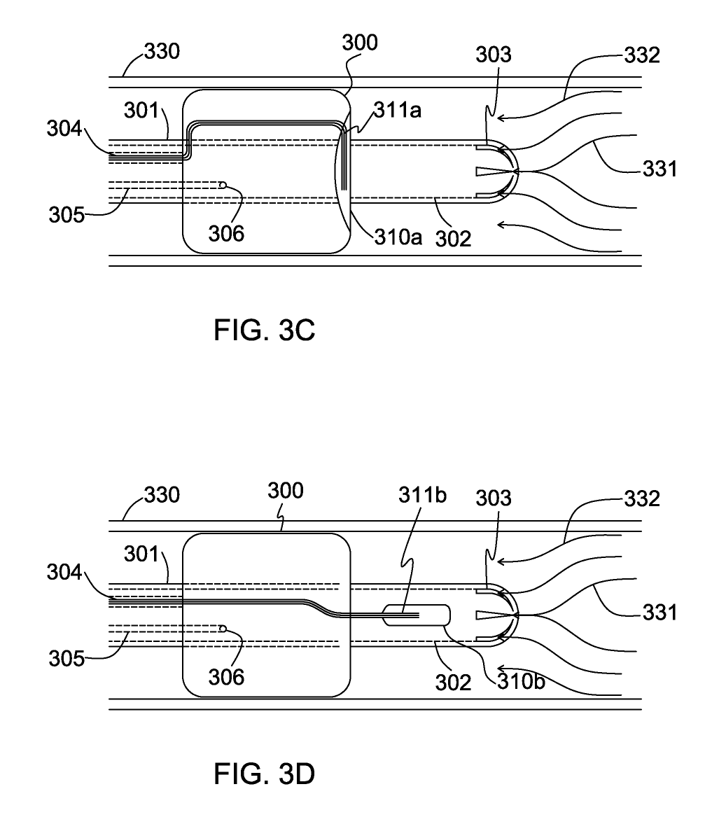

In many embodiments, the sensor portion(s) of the present invention is associated with the balloon portion of the subject catheters and include one or more sensor elements positioned for contact with bodily fluid when positioned within the patient's body. As such, the one or more sensors or sensor elements may be provided on or coupled to an exterior surface of balloon 114, and/or provided on or coupled to an interior surface of balloon 114, and/or provided on or coupled to the catheter's distal tip 116 adjacent the balloon 114. In some embodiments, the one or more sensors are integrated with the balloon material or other material of the system, e.g., extruded with the balloon material, or the like. Such sensor locations may depend on the configuration and/or intended use of the catheter and/or the balloon portion of the subject catheter. For example, the balloon may be configured to expand/inflate to an extent in which its outer surface does not fully contact the vessel or body lumen in which it is finally positioned, as illustrated in FIGS. 2A and 2C. Alternatively, the balloon may be configured to expand/inflate to fully contact or occlude the vessel in which it is positioned, as illustrated in FIGS. 3A, 3C and 3D.

With reference to FIGS. 2A, 2B, and 2C, a balloon 200 of a catheter assembly, for example the catheter assembly of the analyte monitoring system 100 of FIG. 1A, is shown positioned within a bodily vessel 230, e.g., a vein, artery, organ, etc., in which balloon 200 is expanded or inflated to less than the diameter of bodily vessel 230 to allow fluid passage 231 e.g., blood, between the exterior surface of balloon 200 and the interior of vessel wall 230. To facilitate such, balloon 200 may have an elongated configuration with a tapered distal end portion 201, which, in some embodiments, includes an atraumatic distal tip 202 to minimize the chance of tissue damage during delivery of the catheter to the target site within the body. Balloon 200 is inflated via inflation/expansion lumen 205.

Catheter lumen 203 and balloon 200 are designed for fluid passage 231 outside balloon 200, sensors, for example an analyte sensor 210, are provided on the exterior surface of the balloon 200 in this embodiment, but could be located elsewhere. For example, a sensor 210 or sensor element may be provided on the exterior surface of a central or main portion of balloon 200. In an alternative configuration, as illustrated in FIG. 2C, a sensor 210a or sensor element may be provided on a distal portion 201 of balloon 200. The sensor configurations of FIGS. 2A and 2C may be used individually in a single sensor configuration, or may be used on conjunction with one another in a multiple sensor configuration. Where more than one sensor is provided, all may be configured to sense the same analyte or each may be configured differently from the others such that more than one analyte may be sensed using just one catheter, or more than one sensor may be configured to sense the same analyte and one or more sensors may be configured to sense another analyte (or multiple other analytes). The sensors 210 (FIG. 2A) and 210a (FIG. 2C) include electrodes 211 and 211a, respectively, and, in some embodiments, sensor chemistry for sensing target analyte(s). The sensor electrodes 211, 211a extend proximally, either embedded within the balloon material or otherwise insulated along the balloon surface, to electrical contacts or wires which extend through the monitoring lumen 204 to electrically couple with a monitor 104 (FIG. 1A).

Referring now to FIGS. 3A, 3B, 3C, and 3D, another exemplary balloon 300 of a catheter assembly is shown positioned within bodily vessel 330 in which balloon 300 is expanded or inflated to contact the interior wall of bodily vessel 330 thereby occluding the vessel of any fluid flow therethrough. In many circumstances, such as in coronary artery and other cardiac procedures, it is necessary to maintain fluid, i.e., blood, flow distally within the vessel, i.e., downstream of the expanded/inflated balloon. To accomplish such, the balloon 300 of the catheter assembly, is provided with a flow or bypass lumen 301 that extends through balloon 300. The flow lumen 301 additionally may include at least one perfusion lumen 302 open to the exterior of the flow lumen 301 at a first and second locations distal and proximal, i.e., upstream and downstream, relative to the balloon 300, respectively. Here, one or more fluid entry openings 303 are provided distally of balloon 300 to allow entry of the bodily fluid 331 at a distal end of the flow lumen 301. Fluid exit openings or holes (not shown) are provided within flow lumen 301 at one or more locations proximally of balloon 300 to allow fluid 331 to exit lumen 301 back to within the vasculature or organ. In this embodiment, the expansion/inflation lumen 305 terminates distally in a port 306 which extends into the interior space of balloon 300.

Referring still to FIGS. 3A-3D, with bodily fluid prevented from contacting the lateral walls of occlusion balloon 300, a sensor or sensors are provided at locations other than the exterior of the lateral walls of balloon 300. For example, as illustrated in FIGS. 3A and 3B, an analyte sensor 310 may be provided on the interior wall of flow lumen 301 with the sensor electrodes 311 extending directly into a monitoring lumen 304 of the catheter assembly or within the wall of flow lumen 301. Alternatively as illustrated in FIG. 3C, or additionally, a sensor 310a may be provided on an exterior distal end of balloon 300 for contact with residual fluid 332 which is not caused to enter into flow lumen 301 through flow holes 303. As such, the electrodes 311a of sensor 310a may be configured to extend distally within or along the balloon material to a respective monitoring lumen (not shown) within the proximal portion of the catheter assembly or within the wall of flow lumen 301. Still yet, in another configuration illustrated in FIG. 3D, an analyte sensor 310b may be provided on the exterior surface of the distal portion of the catheter assembly with its sensor electrodes 311b extending through a designated lumen (not shown) of the catheter assembly or within the wall of flow lumen 301. The sensor configurations illustrated in FIGS. 3A, 3C and 3D may be used individually in a single sensor configuration, or may be used in conjunction with one another in a multiple sensor configuration. As mentioned above, the plurality of sensors 310, 310a, 310b may be used to take separate measurements of only a single analyte or may each have its own particular sensor chemistry to sense different analytes. In the former embodiment, analyte information obtained by a first sensor may be employed as a comparison to analyte information obtained by a second sensor. This may be useful to confirm or validate analyte information obtained from one or both of the sensors. Such redundancy may be useful if analyte information is contemplated in critical therapy-related decisions.

The target location within the body at which the subject sensors are to be positioned depends, at least in part, on the type of analyte or blood chemistry to be monitored and on the medical condition of the patient. Depending on these circumstances, it may be preferable to monitor the target analyte(s) within a peripheral vein or artery, or at or within a vital organ, e.g., within a heart chamber, the kidney, etc., or within a blood vessel within or near an organ, e.g., vena cava, coronary artery, renal artery, etc. Such vessel and/or organ may or may not be the targeted structure of an underlying surgical or diagnostic procedure. In turn, then, the target location at which the analyte sensor is placed is a consideration in the structure of the subject catheter and balloon to be used, e.g., occlusion, perfusion, etc. as well as in the positioning of the analyte sensor relative to the balloon.

As mentioned above, the subject analyte sensing catheters, catheter assemblies and catheter systems may be used prior to, subsequent to or during any therapeutic or diagnostic procedure where such analyte monitoring is helpful or necessary. Where such therapeutic or diagnostic procedure is catheter-based, the subject sensing catheters and assemblies may be provided or used as part of an overall minimally invasive system and procedure. Most commonly, these procedures are coronary or peripheral artery procedures or other cardiac procedures. Examples of such procedures with which the subject inventions may be used include, but are not limited to, percutaneous transluminal coronary angioplasty (PTCA), percutaneous transluminal embolectomy, directional coronary atherectomy (DCA), cardiac tissue ablation to treat atrial fibrillation, and coronary/vascular stenting. Each of these minimally invasive procedures involves introducing a catheter into a major artery through a small arterial puncture made in the groin, upper arm, or neck. With many of them, a balloon catheter is often used, either to perform the underlying surgical procedures, e.g., to open the stenotic blockage during PTCA, or is ancillary to the procedure, e.g., to temporarily control the flow of blood through a vessel or organ during the procedure. With these procedures, the sensing functions of various embodiments of the present disclosure may be integrated into the existing balloon catheter. With procedures in which a balloon catheter is not already employed, a separate balloon catheter or balloon catheter assembly may be adjunctively utilized for the analyte sensing functions.

A few exemplary catheter-based procedures with which the subject embodiments may be employed are now briefly described in greater detail. The particular steps and techniques discussed with respect to each are merely exemplary and not exclusive to additional steps or variations in the techniques.

In percutaneous transluminal coronary angioplasty (PTCA), a balloon is employed to mechanically dilate an obstruction or occlusions in blood vessels, referred to as stenosis. In PTCA, a steerable guidewire is introduced and advanced under fluoroscopic observation into the stenosed artery and past the stenosis. Next, a balloon-tipped catheter is advanced over the guidewire until it is positioned across the stenosed segment. The balloon is then inflated, separating or fracturing the stenosis, to clear the vascular passage and reestablish blood flow therethrough. Once the blockage is opened, the balloon is deflated and removed from the patient. While this procedure is often effective in relieving the symptoms caused by the disease by dilating the blood vessels for a substantial length of time, the balloon itself will occlude the blood vessel while it is inflated within the vessel. Accordingly, a perfusion catheter, such as the catheter of FIGS. 3A and 3B, is often used for PTCA. The shaft is provided with openings that communicate with this additional lumen on opposite sides of the balloon (the sides of the shaft both distal and proximate from the balloon) so that blood will flow though the lumen when the balloon is inflated, reducing the risks to the patient.

In directional coronary atherectomy (DCA), a catheter containing a cutter housed in its distal end is advanced over a guidewire into the stenosed segment. The housing is urged against the stenosis by the inflation of a balloon so that part of the stenotic material intrudes through a window in the side of the housing. Under fluoroscopic observation, the cutter is used to shave away the stenotic material. The shavings are collected in a nosecone of the catheter and withdrawn along with the catheter or flushed out of a flushing lumen running the length of the device. Some examples of existing devices for performing directional coronary atherectomy are disclosed in U.S. Pat. Nos. 5,074,841; 4,669,469; and 4,867,157.

Stenting is a procedure in which a wire framework, known as a stent, is compressed over a balloon catheter and delivered in the compressed state to a stenosed segment of an artery. Once at the desired location, the balloon is inflated, causing the stent to dilate and forcing it against the artery wall. Once the therapeutic result is achieved the balloon is deflated and removed from the patient, leaving the stent implanted. Frequently, a stent is placed in an artery immediately following PTCA or DCA.

Examples of balloon catheters and their applications with which the aspects of the present disclosure may be employed are disclosed in U.S. Pat. Nos. 5,176,661; 5,242,394; 5,242,396; 5,290,230; 5,300,025; 5,300,085; 5,316,706; 5,324,259; 5,344,426; 5,346,505; 5,348,537; 5,411,476; 5,423,755; 5,451,209; 5,451,233; 5,458,613; 5,480,383; 5,496,275; 5,496,346; 5,525,388; 5,533,968; 5,542,925; 5,554,120; 5,554,121; 5,565,523; 5,743,875; 5,749,888; 5,769,868; 5,816,923; 5,830,181; 5,849,846; 5,868,706; 5,891,090; 5,902,290; 5,931,819; 5,989,218; 5,993,460; 6,013,054; 6,013,069; 6,013,728; 6,019,777; 6,027,475; 6,036,707; 6,059,748; 6,061,588; 6,117,106; 6,126,634; 6,126,635; 6,129,707; 6,136,011; 6,139,525; 6,156,047; 6,165,152; 6,165,292; 6,179,810; 6,193,686; 6,206,852; 6,217,547; 6,221,425; 6,224,803; 6,238,376; 6,248,092; 6,251,094; 6,368,301; 6,488,688; 6,561,788; 6,572,813; 6,579,484; 6,663,614; 6,575,993; 6,589,207; 6,835,059; and 7,273,487; the entireties of which are incorporated herein by reference for all purposes.

The methods of the present disclosure include one or more of the mechanical activities associated with use of the subject devices described above, and may additionally include any of the steps or activities involved in the performance of any therapeutic or diagnostic procedure, including any minimally invasive or catheter-based procedures, not limited to those exemplary procedures described briefly above or disclosed in the cited patent references. Further, the steps or activities involving the subject devices may be collectively performed prior to, during or subsequent to such procedures. Certain steps or activities involved in using the subject devices may be performed once or repeatedly at various intervals throughout the underlying therapeutic or diagnostic procedure. Any particular sequence of steps or activities that have been described above and/or claimed below have been selected and so ordered for typographical convenience and are not intended to imply any particular order for performing the subject methods.

The subject devices, systems and methods may be configured and employed to continuously measure or monitor one or more selected analytes or any other physiological chemistry present in bodily fluid. Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times. Examples of particular analyte sensor designs and systems suitable for use with the present invention are disclosed in U.S. Pat. Nos. 5,262,035; 5,264,104; 5,262,305; 5,320,715; 5,593,852; 6,175,752; 6,650,471; 6,746,582, and in U.S. Patent Application Publication No. 2004/0186365, each of which is incorporated herein by reference. A number of these in vivo systems are based on “enzyme electrode” technology, whereby an enzymatic reaction involving an enzyme such as glucose oxidase, glucose dehydrogenase, or the like, is combined with an electrochemical sensor for the determination of an individual's glucose level in a sample of the individual's biological fluid.

In another aspect of this invention, the subject sensors are adapted to continuously measure or monitor biological chemistries other than analytes, including, for example, arterial blood gases (ABG). Arterial blood gas values such as O₂, CO₂, NO₂ and pH are several of the most frequently ordered laboratory examinations in the intensive care setting and the operating room. In the intensive care unit (ICU), ABG is typically monitored once a day and additional measurements are only made once the patient has experienced a deleterious event. Limited additional sampling is performed at the discretion of a physician or nurse. There can be a significant time delay between the time the tests are ordered and the time at which the results are returned. With the continuous monitoring provided by the present invention, potentially catastrophic events signaled by rapid changes in ABGs could be avoided.

In one embodiment, a device for in vivo monitoring of one or more selected analytes within a patient may comprise a catheter comprising a balloon at a distal portion of the catheter, and at least one analyte sensor associated with the balloon, wherein the at least one analyte sensor is positioned for contact with a bodily fluid when the balloon is operatively deployed within the patient.

An analyte sensor may be provided on an exterior surface of the balloon.

An analyte sensor may be provided on an interior surface of the balloon.

An analyte sensor may be provided on the distal portion of the catheter adjacent the balloon.

The distal portion of the catheter may comprise at least one opening therein for the ingress of bodily fluid therethrough.

A portion of the catheter proximal to the balloon may comprise at least one opening therein for the egress of bodily fluid therethrough.

At least one analyte sensor may be a glucose sensor.

In one aspect, the catheter may be a multi-lumen catheter comprising at least one balloon expansion lumen and at least one lumen for the delivery of an instrument for performing a medical procedure.

The catheter may be configured for performing a therapeutic procedure.

The catheter may be configured for performing a diagnostic procedure.

The catheter may be configured for performing an intravascular procedure.

The procedure may comprise at least one of percutaneous transluminal coronary angioplasty, stent placement, directional coronary atherectomy and cardiac tissue ablation.

The balloon may be configured for expansion against a tissue structure within the patient.

In another embodiment, catheter system for performing a procedure within the body may comprise a balloon catheter comprising at least one analyte sensor associated with a balloon provided at a distal portion of the catheter, and an analyte monitor in communication with the at least one analyte sensor.

One aspect may include at least one tool for delivery through a lumen of the catheter to a target location within the body.

At least one tool may be a guide wire.

Another aspect may include a wire connection between the analyte monitor and the analyte sensor.

Yet another aspect may include a wireless connection between the analyte monitor and the analyte sensor.

In another embodiment, a catheter system for performing a procedure within the body may comprise an analyte sensing catheter comprising at least one analyte sensor associated with a balloon provided at a distal portion of the catheter, and at least a second catheter configured for performing a medical procedure at a target location within the body.

Furthermore, the at least the second catheter may be configured for performing a diagnostic procedure.

Moreover, the at least the second catheter may be configured for performing a therapeutic procedure.

In another embodiment of the present disclosure, a method for continuously measuring at least one target analyte within the body of a patient may comprise providing a catheter comprising at least one analyte sensor associated with a balloon provided at a distal portion of the catheter, delivering the catheter to a location in the body and expanding the balloon at the location wherein the at least one analyte sensor is positioned for contact with a bodily fluid, and monitoring at least one target analyte in the bodily fluid for a period of time.

Furthermore, the method may include performing a medical procedure within the body during at least a portion of the period of time the at least one target analyte is monitored.

Monitoring the at least one target analyte may comprise determining when the at least one target analyte is within a safe range.

Moreover, the method may include performing the medical procedure when the at least one target analyte is within the safe range.

The medical procedure may comprise employing the catheter to perform at least a portion of the procedure.

The medical procedure may be performed proximate the location in the body at which the balloon is expanded.

The medical procedure may comprise at least one of percutaneous transluminal coronary angioplasty, stent placement, directional coronary atherectomy, or cardiac tissue ablation.

As for other details of the present disclosure, materials and alternate related configurations may be employed as within the level of those with skill in the relevant art. The same may hold true with respect to method-based aspects of the disclosure in terms of additional acts as commonly or logically employed. In addition, though the embodiments of the present disclosure have been described in reference to several examples, optionally incorporating various features, the invention is not to be limited to that which is described or indicated as contemplated with respect to each variation of the invention. Various changes may be made to the embodiments described, and equivalents (whether recited herein or not included for the sake of some brevity) may be substituted without departing from the true spirit and scope of the present disclosure. Any number of the individual parts or subassemblies shown may be integrated in their design. Such changes or others may be undertaken or guided by the principles of design for assembly.

Also, it is contemplated that any optional feature of the inventive variations described may be set forth and claimed independently, or in combination with any one or more of the features described herein. As used herein and in the appended claims, the singular forms “a,” “and,” and “the” include plural referents unless the context clearly dictates otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Stated otherwise, unless specifically defined herein, all technical and scientific terms used herein are to be given as broad a commonly understood meaning as possible while maintaining claim validity.

What is claimed is:

1. A device for in vivo monitoring of one or more selected analytes within a patient, the device comprising:
a catheter comprising a balloon at a distal portion of the catheter; and
at least one analyte sensor associated with the balloon, wherein the at least one analyte sensor is positioned for contact with a bodily fluid when the balloon is operatively deployed within the patient.

2. The device of claim 1 wherein an analyte sensor is provided on an exterior surface of the balloon.

3. The device of claim 1 wherein an analyte sensor is provided on an interior surface of the balloon.

4. The device of claim 1 wherein an analyte sensor is provided on the distal portion of the catheter adjacent the balloon.

5. The device of claim 1 wherein the distal portion of the catheter comprises at least one opening therein for the ingress of bodily fluid therethrough.

6. The device of claim 5 wherein a portion of the catheter proximal to the balloon comprises at least one opening therein for the egress of bodily fluid therethrough.

7. The device of claim 1 wherein at least one analyte sensor is a glucose sensor.

8. The device of claim 1 wherein the catheter is a multi-lumen catheter comprising at least one balloon expansion lumen and at least one lumen for the delivery of an instrument for performing a medical procedure.

9. The device of claim 1 wherein the catheter is configured for performing a therapeutic procedure.

10. The device of claim 1 wherein the catheter is configured for performing a diagnostic procedure.

11. The device of claim 1 wherein the catheter is configured for performing an intravascular procedure.

12. The device of claim 9 wherein the procedure comprises at least one of percutaneous transluminal coronary angioplasty, stent placement, directional coronary atherectomy and cardiac tissue ablation.

13. The device of claim 1 wherein the balloon is configured for expansion against a tissue structure within the patient.

14. A catheter system for performing a procedure within the body, the system comprising:
a balloon catheter comprising at least one analyte sensor associated with a balloon provided at a distal portion of the catheter; and
an analyte monitor in communication with the at least one analyte sensor.

15. The catheter system of claim 14 further comprising at least one tool for delivery through a lumen of the catheter to a target location within the body.

16. The catheter system of claim 15 wherein at least one tool is a guide wire.

17. The catheter system of claim 14 further comprising a wire connection between the analyte monitor and the analyte sensor.

18. The catheter system of claim 14 further comprising a wireless connection between the analyte monitor and the analyte sensor.

19. A catheter system for performing a procedure within the body, the system comprising:
an analyte sensing catheter comprising at least one analyte sensor associated with a balloon provided at a distal portion of the catheter; and
at least a second catheter configured for performing a medical procedure at a target location within the body.

20. The catheter system of claim 19 wherein the at least the second catheter is configured for performing a diagnostic procedure.

21. The catheter system of claim 19 wherein the at least the second catheter is configured for performing a therapeutic procedure.

22. A method for continuously measuring at least one target analyte within the body of a patient, the method comprising:
providing a catheter comprising at least one analyte sensor associated with a balloon provided at a distal portion of the catheter;
delivering the catheter to a location in the body and expanding the balloon at the location wherein the at least one analyte sensor is positioned for contact with a bodily fluid; and
monitoring at least one target analyte in the bodily fluid for a period of time.

23. The method of claim 22 further comprising performing a medical procedure within the body during at least a portion of the period of time the at least one target analyte is monitored.

24. The method of claim 23 wherein monitoring the at least one target analyte comprises determining when the at least one target analyte is within a safe range.

25. The method of claim 24 further comprising performing the medical procedure when the at least one target analyte is within the safe range.

26. The method of claim 23 wherein the medical procedure comprises employing the catheter to perform at least a portion of the procedure.

27. The method of claim 23 wherein the medical procedure is performed proximate the location in the body at which the balloon is expanded.

28. The method of claim 23 wherein the medical procedure comprises at least one of percutaneous transluminal coronary angioplasty, stent placement, directional coronary atherectomy, or cardiac tissue ablation.
